(19) United States
(12) Patent Application Publication
(10) Pub. No.: US 2014/0172013 A1
(43) Pub. Date: Jun. 19, 2014

(54) Vascular Closure Devices

(71) Applicant: ECD MEDICAL, San Clemente, CA (US)

(72) Inventors: Fred Burbank, Laguna Niguel, CA (US); Michael Jones, San Clemente, CA (US); R. J. Serra, Laguna Hills, CA (US)

(73) Assignee: ECD MEDICAL, San Clemente, CA (US)

(21) Appl. No.: 14/105,494

(22) Filed: Dec. 13, 2013

Related U.S. Application Data
(60) Provisional application No. 61/737,323, filed on Dec. 14, 2012.

(51) Int. Cl. A61B 17/00

(52) U.S. Cl. CPC A61B 17/0057
USPC 606/213

(57) ABSTRACT

A vascular closure device is formed of bioresorbable materials that decompose in vivo at different rates.

### BACKGROUND

1. Field of Endeavor

The present invention relates to devices, systems, and processes useful as closure devices for arterial punctures.

2. Brief Description of the Related Art

Numerous vascular closure devices have been developed over the years. The most utilized one to date is the Abbott Vascular Perclose device. This device utilizes sutures through the wall of the arteriotomy to dose the vessel after removal of the sheath.

Numerous other devices are on the market and have been tried clinically. These range from clips applied to the exterior of the vessel to perform like a suture through plugs inserted into the vessel, as well as some adhesives applied to the vessel.

Other prior devices include the Abbott Vascular Starclose, St June Angio-Seal, Access Closure Mynx, Medtronic EVS Vascular Staple, Medafor Mpatch, Sutura Super Stitch, Vascular Solutions Duett, and Cordis ExoSeal.

### SUMMARY

According to a first aspect of the subject matter described in this application, a bioresorbable vascular closure device comprises a sleeve formed of a bioresorbable material, a catch arm formed of a bioresorbable material, and a locking sleeve formed of a bioresorbable material, wherein the sleeve bioresorbable material, the catch arm bioresorbable material, and the locking sleeve bioresorbable material are mutually selected to degrade in vivo at at least two different rates.

According to a second aspect of the subject matter described in this application, a method of maintaining access to the interior of a blood vessel of a patient comprises positioning a hollow sleeve over an opening formed in the blood vessel, positioning a pair of arms around the sleeve and around the blood vessel, each of the arms including a hook on a lower end thereof, and positioning a locking sleeve over the hollow sleeve and the pair of arms, the locking sleeve forcing the pair of arms radially inwardly and the hooks to ensnare the blood vessel.

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawings, in which:

FIG. 1 illustrates a perspective view of an exemplary system in a blood vessel B, with several portions in a retracted position;

FIG. 2 illustrates another perspective view of an exemplary system in a blood vessel B, similar to that of FIG. 1, with an introducer withdrawn;

FIG. 3 illustrates another perspective view of an exemplary system in a blood vessel B, with an exemplary sleeve positioned on the blood vessel;

FIG. 4 illustrates another perspective view of an exemplary system in a blood vessel B, with an exemplary catch arm positioned over the sleeve;

FIG. 5 illustrates another perspective view of an exemplary system in a blood vessel B, with an exemplary locking sleeve positioned over the catch arm and sleeve;

FIG. 6 illustrates another perspective view of an exemplary system in a blood vessel B, with the sleeve withdrawn;

FIG. 7 illustrates a side elevational view of an exemplary system in a blood vessel B, similar to FIGS. 1;

FIG. 8 illustrates a side elevational view of an exemplary system in a blood vessel B, similar to FIG. 2;

FIG. 9 illustrates a side elevational view of an exemplary system in a blood vessel B, similar to FIG. 3;

FIG. 10 illustrates a side elevational view of an exemplary system in a blood vessel B, similar to FIG. 4;

FIG. 11 illustrates a side elevational view of an exemplary system in a blood vessel B, similar to FIG. 5;

FIG. 12 illustrates a side elevational view of an exemplary system in a blood vessel B, similar to FIG. 6;

FIG. 13 illustrates a perspective view of an exemplary catch arm;

FIG. 14 illustrates a perspective view of an exemplary sleeve; and

FIG. 15 illustrates a perspective view of an exemplary locking sleeve.

### DETAILED DESCRIPTION OF EXEMPLARY EMBODIMENTS

Referring to the drawing figures, like reference numerals designate identical or corresponding elements throughout the several figures.

Devices and processes embodying principles of the present invention capitalize on the benefits of resorbable materials, such as those described in U.S. application Ser. No. 61/737,272, by Fred Burbank and Michael Jones, filed on even date herewith, Attorney Ref. No. 099-001P, entitled Short Term Biomaterials, the entirety of which is incorporated by reference herein. These novel compositions and designs perform the closure of the vessel arteriotomy with an external (to the vessel) system much like the placing of a valve or patch onto a tire.

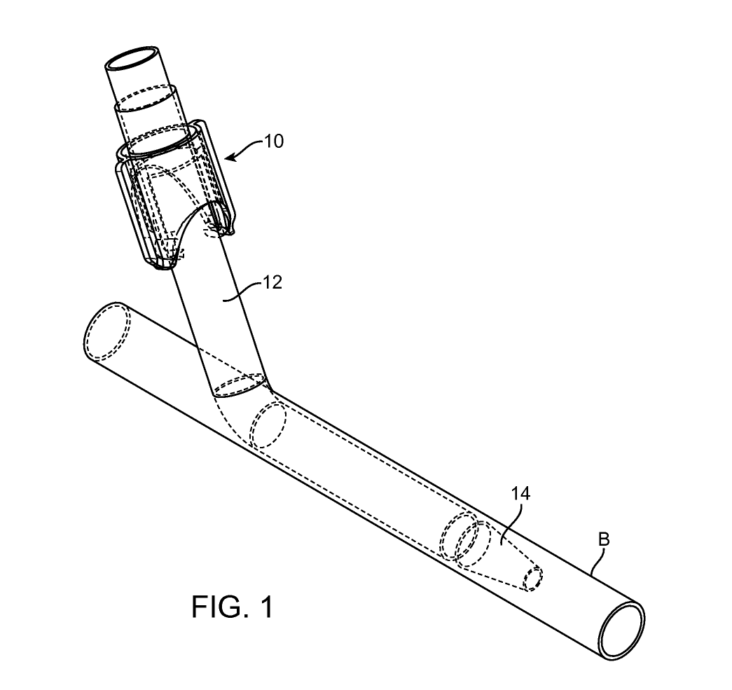

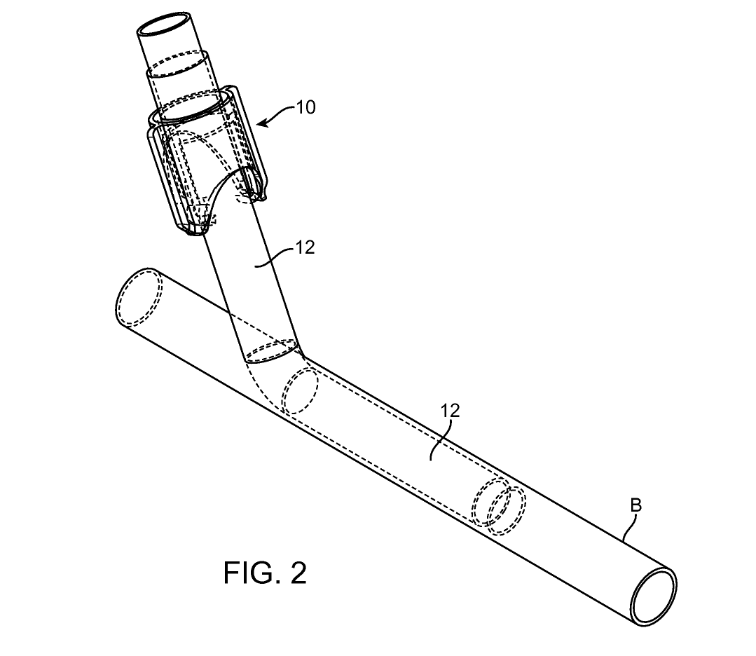

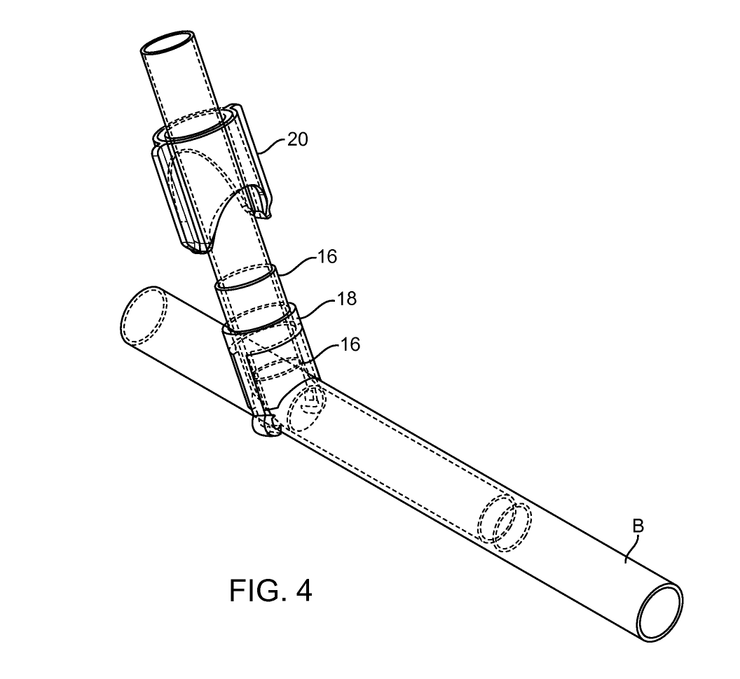

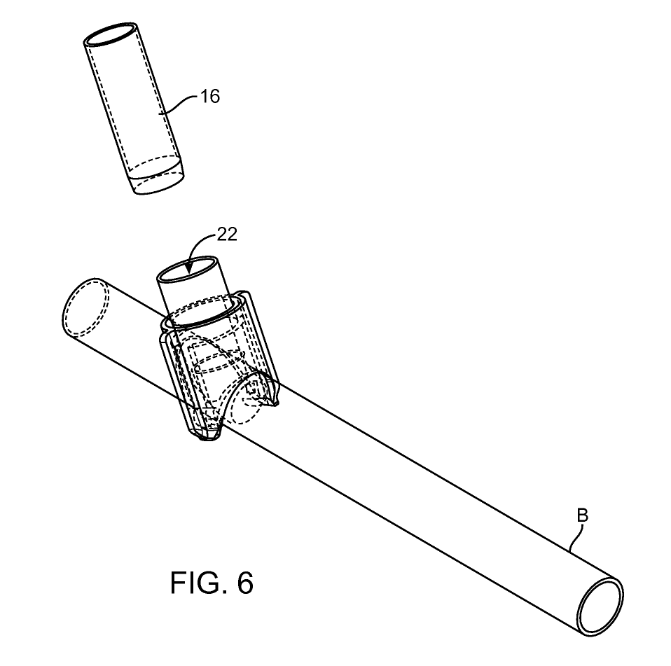

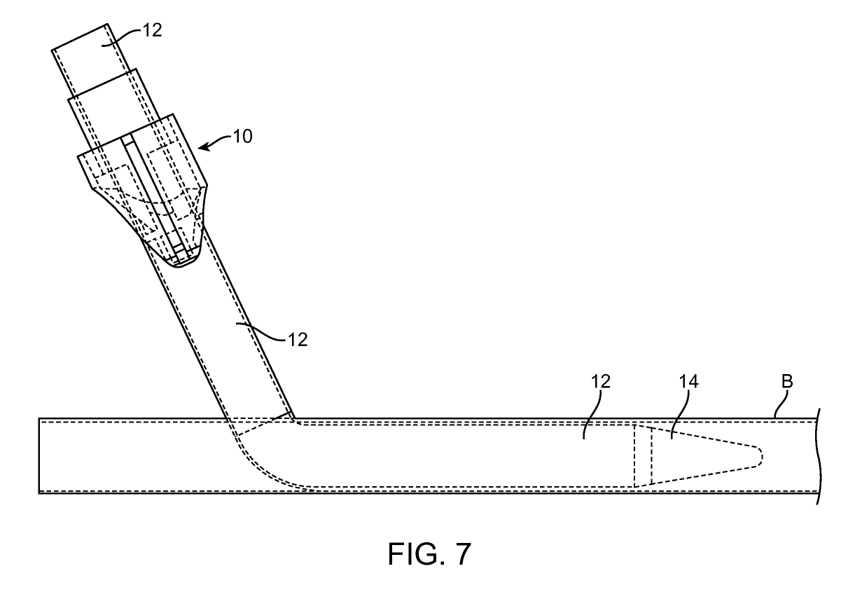

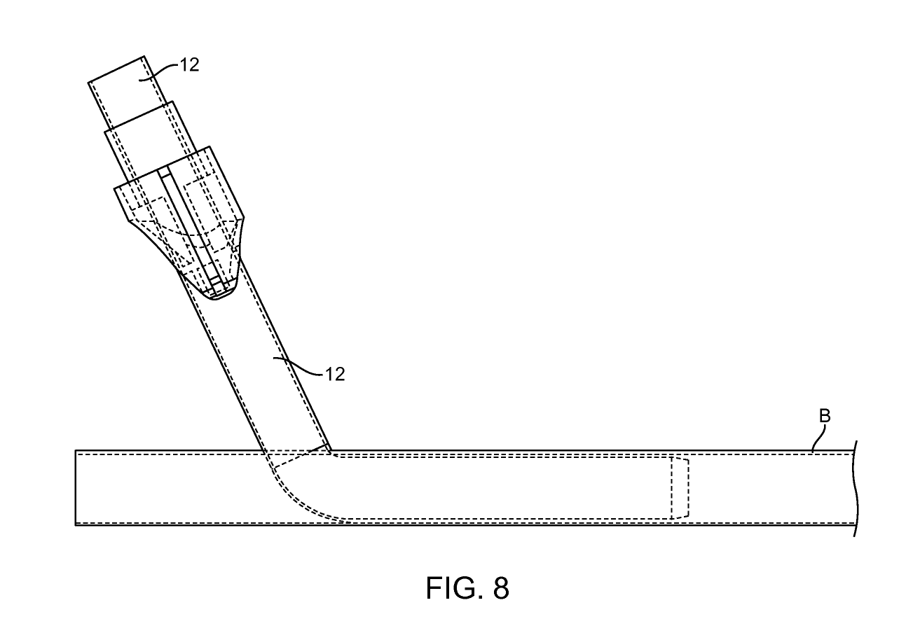

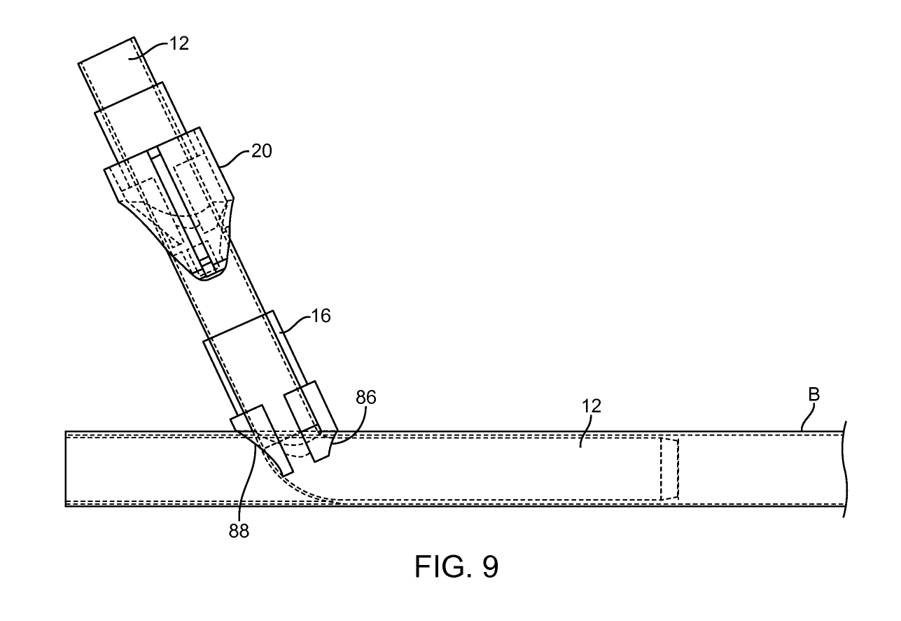

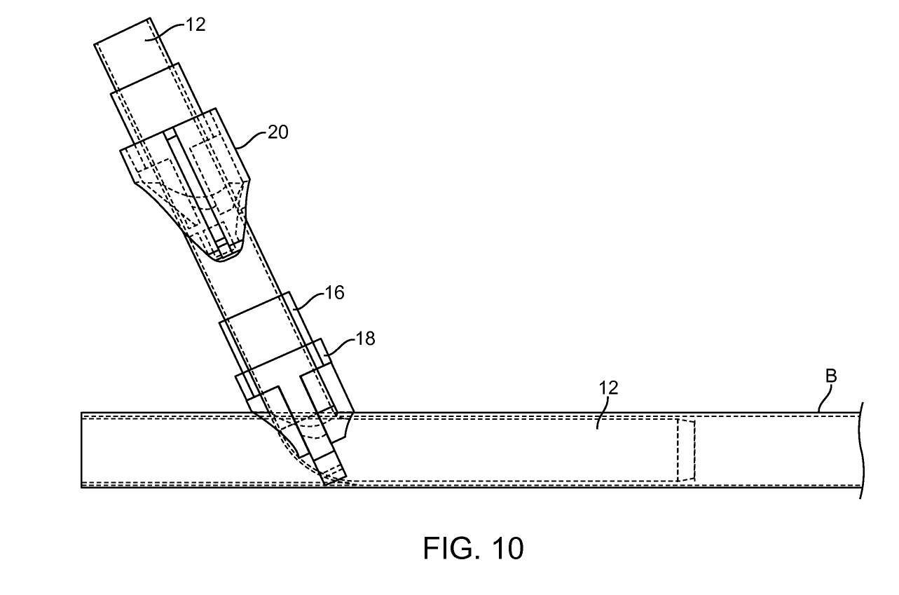

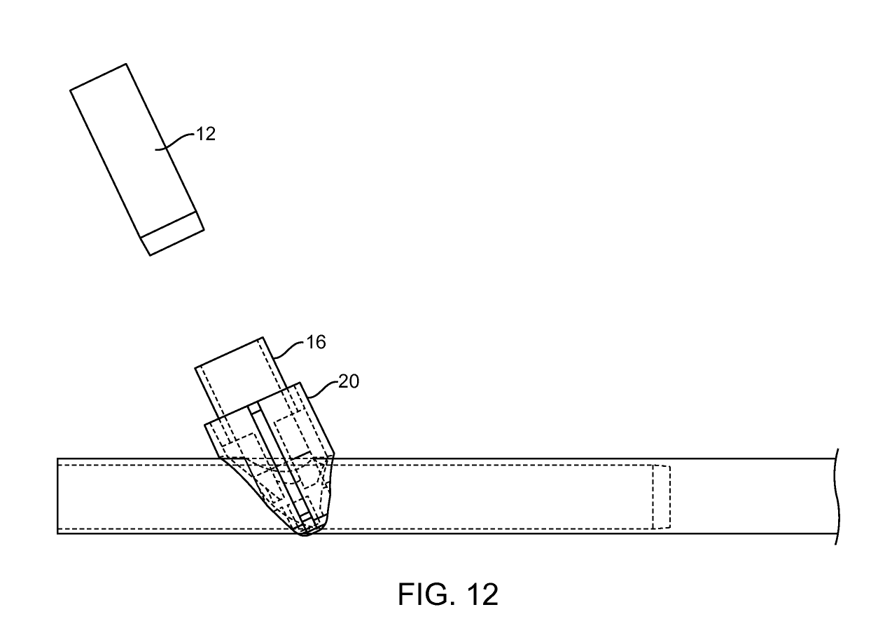

A series of parts of a vascular closure assembly 10, tailored to match the outer diameter (OD) of the blood vessel B, is assembled over a vascular sheath 12 and introducer 14, as illustrated in sequence in FIGS. 1-6 (and, correspondingly, FIGS. 7-12). Once vascular access is obtained, e.g., via an arteriotomy, the parts are slid down the sheath 12 and onto the artery B.

A sleeve 16 is the first item to be placed against the artery. It includes a saddle cut, described in greater detail elsewhere herein, that allows the sleeve to sit down on the artery and eventually seal around the periphery of the arteriotomy.

Next, a catch arm 18 is slid down the introducer and onto the sleeve. This piece nests onto the sleeve and prongs on the ends of arms of the catch arm grasp and retain the artery. Optionally, a locking sleeve 20 can be slid down over the catch arm to lock this piece in place onto the artery.

Once all of these items are placed on top of the artery, a biological sealant can be applied to the base of sleeve to seal the sleeve to the artery. This material can be a bioadhesive such as TISSEAL, BIOGLUE, or a cyanoacrylate. Alternately, a filler such as chitosan, which is a liquid and gels upon a change in pH, or other biologically acceptable hydrogel, could be used to establish a leak-free connection between the sleeve and the artery.

When the interventional procedure is completed, the catheter and access sheath can be removed and a resorbable plug, such as one made from compressed starch, may be inserted into the sleeve to prevent the backflow of blood, e.g., to act like a cork in a wine bottle. Alternately, the catheter could be removed from the sheath, then the sheath backed out into the sleeve and an expandable plug inserted through the sheath into the sleeve, or flowable material that gels in the sleeve and left in place before removing the sheath from the patient.

Alternate embodiments are envisioned that would use a structure allowing for an increase in surface area in the area of bonding of the sleeve to the artery. These embodiments may not require the catch arms to be utilized and instead will utilize a tissue adhesive and larger surface contact area to provide the seal and bond.

Particularly advantageously, these constructions are formed of a material or materials that is resorbed by the body in a short time span, ranging from 24 to 96 hours, leaving an organized clot as the closure of the vessel.

According to a first exemplary embodiment, the sleeve is formed from a compressed composition of starch with 20% (by weight percent) methyl cellulose mixed as a binder. This material, when compressed at about 40,000 to 50,000 psi, become a useable solid material that has very good compressive strength, but little tensile strength. The catch arms can be made of a moldable composition such as 65/35 copolymer of poly-lactic and poly-glycolic acid. This copolymer may be constructed with a short-term filler, such as starch or other polysaccharide, to accelerate its decomposition in vivo. The locking sleeve is formed from a similar material as the catch arms as it will experience some tensile load as it slides down and locks onto the catch arms. As will be immediately appreciated, alternate materials are possible, while the material construction described above is a preferred embodiment.

Devices as described herein can have numerous advantages over prior closure devices. Devices embodying principles of the present invention work well with small arteriotomies that accompany small introducers in the 5 to 7 Fr range and work adequately on larger introducers up to 9Fr. Another benefit is that nothing is left within the artery as a nidus for clot formation or narrowing within the vessel. Additionally, there will be nothing left long term to cause the artery to narrow at the entry site due to exterior inflammation.

### EXAMPLES

I. The sleeve is molded from a bioresorbable polymer such as 63/35 PLGA with resorption time of 6-8 weeks in vivo. The catch arm is molded from a bioresorbable polymer such as 63/35 PLGA with resorption time of 6-8 weeks in vivo. The locking sleeve is molded from a bioresorbable polymer such as 63/35 PLGA with resorption time of 6-8 weeks in vivo. The hemostatic plug is made from a compressed starch with 20% by weight methyl cellulose as a binder. This plug will be resorbed in the body in about 4-7 days.

II. The sleeve is compression molded from a bioresorbable, hemostatic starch with 20% by weight methyl cellulose as a binder. The catch arm is molded from a bioresorbable polymer such as 63/35 PLGA with resorption time of 6-8 weeks in vivo. The locking sleeve is molded from a bioresorbable polymer such as 63/35 PLGA with resorption time of 6-8 weeks in vivo. The hemostatic plug is made from a compressed starch with 20% by weight methyl cellulose as a binder. This plug will be resorbed in the body in about 4-7 days.

III. The sleeve is compression molded from a bioresorbable, hemostatic chitosan with 20% by weight methyl cellulose as a binder. The catch arm is molded from a bioresorbable polymer such as 63/35 PLGA with resorption time of 6-8 weeks in vivo. The locking sleeve is molded from a bioresorbable polymer such as 63/35 PLGA with resorption time of 6-8 weeks in vivo. The hemostatic plug is made from a compressed starch with 20% by weight methyl cellulose as a binder. This plug will be resorbed in the body in about 4-7 days.

IV. The sleeve is formed from a freeze dried bioresorbable, hemostatic chitosan. The catch arm is molded from a bioresorbable polymer such as 63/35 PLGA with resorption time of 6-8 weeks in vivo. The locking sleeve is molded from a bioresorbable polymer such as 63/35 PLGA with resorption time of 6-8 weeks in vivo. The hemostatic plug is made from a compressed starch with 20% by weight methyl cellulose as a binder. This plug will be resorbed in the body in about 4-7 days.

V. The sleeve is molded from a bioresorbable polymer composite containing 20 to 50% by weight starch in a 63/35 PLGA with resorption time of 4-6 weeks in vivo. The catch arm is molded from a bioresorbable polymer composite containing 20 to 50% by weight starch in a 63/35 PLGA. The locking sleeve is molded from a bioresorbable polymer composite containing 20 to 50% by weight starch in a 63/35 PLGA. The hemostatic plug is made from a compressed starch with 20% by weight methyl cellulose as a binder. This plug will be resorbed in the body in about 4-7 days.

VI. The sleeve is molded from a bioresorbable polymer composite containing 20 to 50% by weight chitosan in a 63/35 PLGA with resorption time of 4-6 weeks in vivo. The catch arm is molded from a bioresorbable polymer composite containing 20 to 50% by weight starch in a 63/35 PLGA. The locking sleeve is molded from a bioresorbable polymer composite containing 20 to 50% by weight starch in a 63/35 PLGA. The hemostatic plug is made from a compressed starch with 20% by weight methyl cellulose as a binder. This plug will be resorbed in the body in about 4-7 days.

VII. The sleeve is formed from a freeze dried bioresorbable composite, of 20 to 50% by weight starch in chitosan. The catch arm is molded from a bioresorbable polymer composite of 20 to 50% starch in 63/35 PLGA with resorption time of 4-6 weeks in vivo. The locking sleeve is molded from a bioresorbable polymer composite of 20 to 50% starch in 63/35 PLGA with resorption time of 4-6 weeks in vivo. The hemostatic plug is made from a compressed starch with 20% by weight methyl cellulose as a binder. This plug will be resorbed in the body in about 4-7 days.

Other uses of include vascular graft or fistula creation for use in kidney dialysis patients. In these patients, a graft is created between an artery and vein in the arm to allow for easy access with two cannulae for dialysis. In this use, there would be two of these connectors, one positioned on the artery and another positioned on the vein with a graft synthetic or a harvested vein extending between the two. This would allow for optimization of the graft takeoff and return angle to minimize fluid flow disruption at the graft and re-entry site. Additionally, with no suture in the inner lumen of the vessel, clotting would be less of a problem than with current solutions.

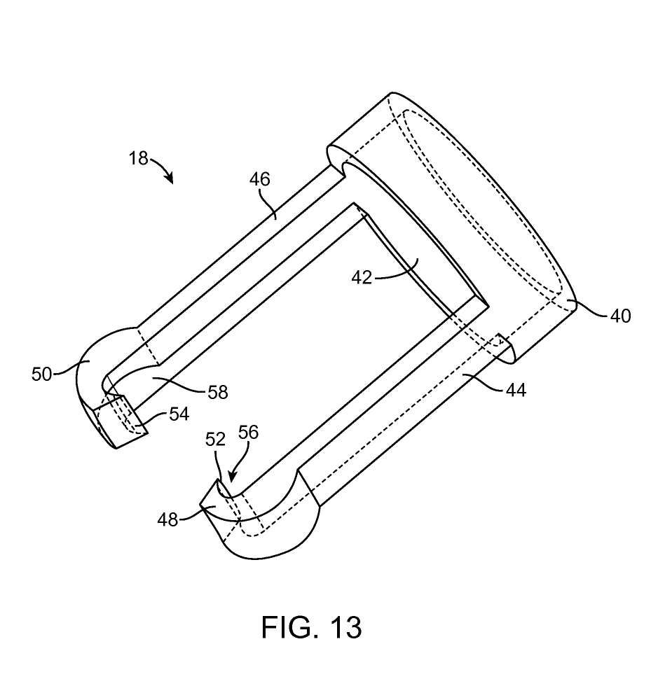

Turning now to FIG. 13, and exemplary catch arm 18 is illustrated in a perspective view. The catch arm includes an upper cylindrical portion 40 having a hollow interior space 42, sized so that the catch arm 18 can slide over the exterior of the sheath 12. A pair of arms 44, 46 extend downward from the portion 40, advantageously diametrically opposed to each other, a distance sufficient for the lower ends of the arms 44, 46 to extend around a blood vessel B, as described elsewhere herein. Each arm 44, 46 includes a hooked lower end 48, 50; in the exemplary embodiment illustrated, the hooks are generally C-shaped, although other configurations of hooks can perform well. At a radially inner end, each end 48, 50 includes a pointed tip 52, 54, each directed inwardly and advantageously somewhat towards the portion 40, so that the arms 44, 46 can both surround and ensnare a blood vessel B therebetween. Recesses 56, 58 and thus formed between the ends 48, 50, and the adjacent portions of the arms.

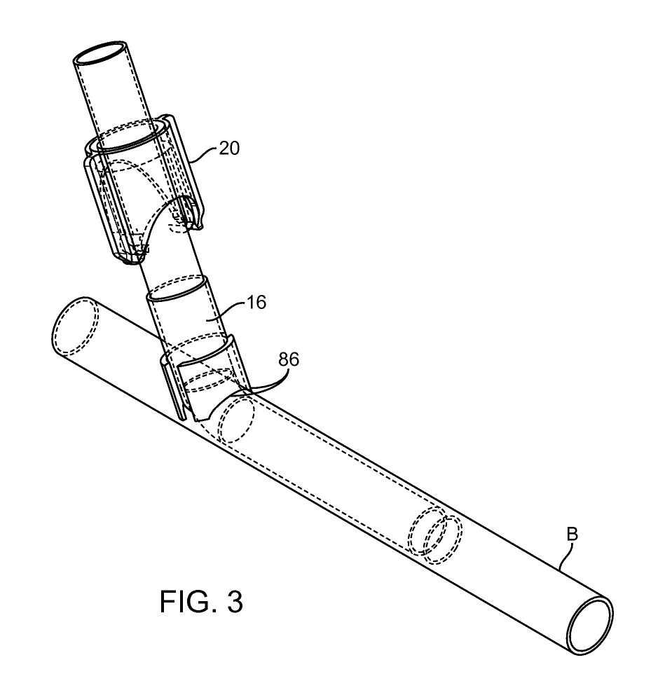

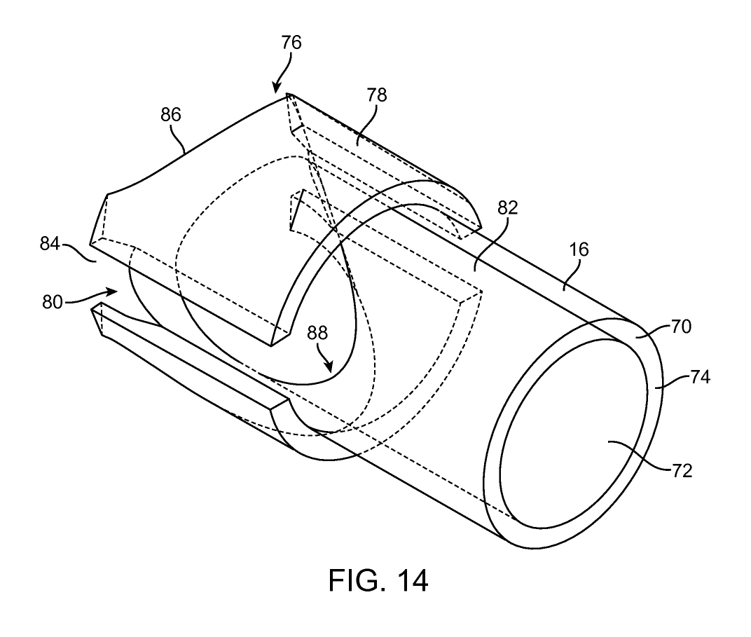

Turning now to FIG. 14, and exemplary sleeve 16 is illustrated in a perspective view. The sleeve 16 generally includes two portions: a hollow cylindrical portion 70; and a saddle portion 78 formed integrally therewith. As discussed elsewhere herein, the sleeve 16 is sized so that, when placed over a vascular access point in a blood vessel B, the sleeve can assist in stabilizing the access point and provide a structure that can be attached, e.g., adhered, to the blood vessel. The sleeve includes a hollow interior 72, an upper end 74, and a lower end 76 opposite the upper end. The saddle portion 78 is formed at the lower end of the sleeve 16, an extends radially outward from the diameter of the cylindrical portion 70, so other portions of the assembly (e.g., a catch arm and an optional locking sleeve) can slide over the outer surface of the sleeve, as illustrated and described herein. The saddle portion 78 includes a pair of open slots 80, 82, sized, configured, and oriented so that the arms 44, 46 can slide down the slots 80, 82, as the catch arm 18 is slid over the cylindrical portion 70. The slots 80, 82 end in lower openings 84, which are positioned so that the hooks 52, 54 can extend into the lower openings 84 and ensnare a blood vessel B therebetween, and thus secure the sleeve 16 to the blood vessel. The lower end of the saddle 78 includes curved cutouts 86, 88 (see also FIGS. 3, 4, 9, 10), positioned circumferentially between the slots 80, 82 and opposite each other, with the cutout 86 being shallower than the cutout 88, so the sleeve can be mounted around the approximately cylindrical outer surface of the blood vessel B at an angle, as illustrated herein.

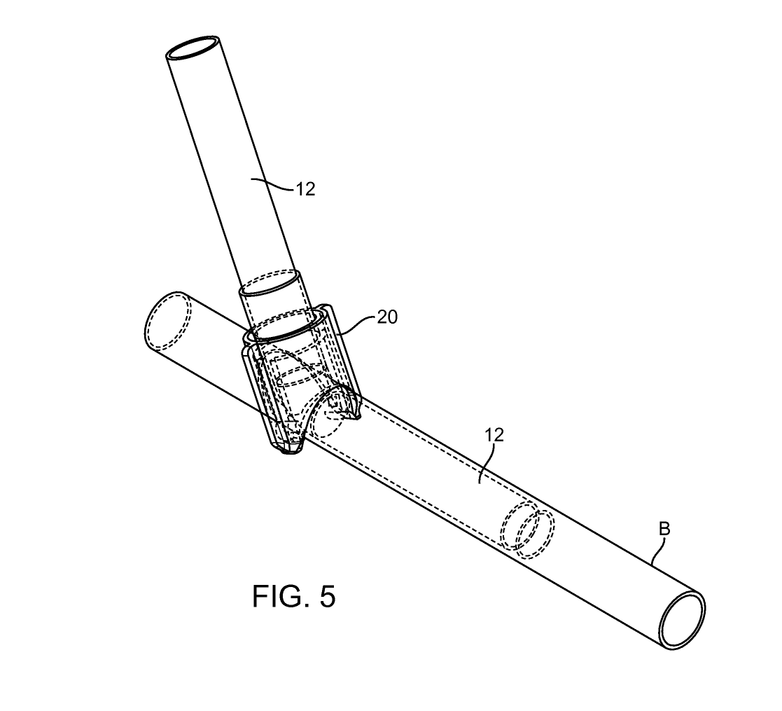

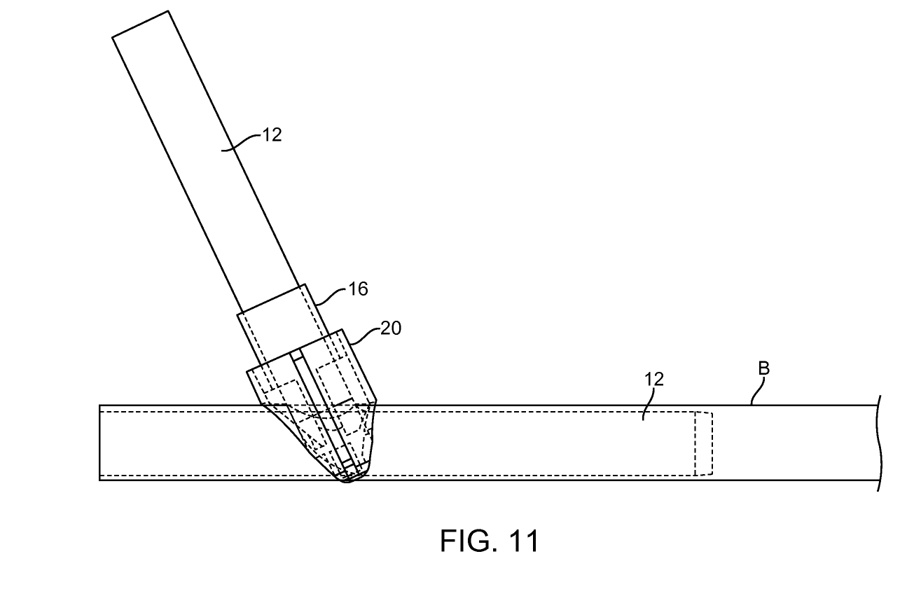

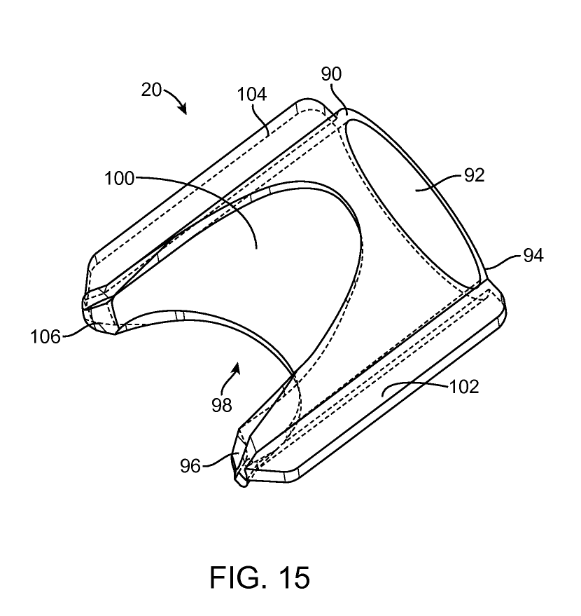

Turning now to FIG. 15, and locking sleeve 20 is illustrated in a perspective view. The locking sleeve 20 includes a generally cylindrical body 90 having a hollow interior 92, sized to slide over the exterior of catch arm 18 and the sleeve 16 (see, e.g., FIGS. 5 and 11). The sleeve 20 includes an upper end 94, a lower end 96, and curved cutouts 98, 100 similar to cutouts 86, 88. The exterior of the cylinder 90 includes a pair of reinforcing ribs 102 that extend along the length of the cylinder and are positioned so that, when the sleeve 20 is positioned over the catch arm 18 and the sleeve 16, overlies the arms 44, 46 and inhibits, and advantageously prevents, the arms from flexing outwardly. The lower ends of the ribs each include an inclined camming surface 106 which is oriented so that the surfaces 106 bear against the outer surfaces of the hooks 48, 50 when the sleeve 20 is pushed over the catch arm 18, forcing the arms 44, 46 radially inwardly to ensnare the blood vessel B therebetween. While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

We claim:

1. A bioresorbable vascular closure device comprising:
a sleeve formed of a bioresorbable material;
a catch arm formed of a bioresorbable material;
a locking sleeve formed of a bioresorbable material;
wherein the sleeve bioresorbable material, the catch arm bioresorbable material, and the locking sleeve bioresorbable material are mutually selected to degrade in vivo at at least two different rates.

2. A bioresorbable vascular closure device according to claim 1, wherein the sleeve bioresorbable material, the catch arm bioresorbable material, and the locking sleeve bioresorbable material are each selected from the group consisting of:
63/35 PLGA;
a polymer composite containing 20 to 50% by weight starch in a 63/35 PLGA;
a bioresorbable, hemostatic chitosan with 20% by weight methyl cellulose as a binder;
a bioresorbable polymer composite containing 20 to 50% by weight starch in a 63/35 PLGA;
a bioresorbable polymer composite containing 20 to 50% by weight chitosan in a 63/35 PLGA;
a freeze dried bioresorbable, hemostatic chitosan;
a freeze dried bioresorbable composite of 20 to 50% by weight starch in chitosan;
a compressed starch with 20% by weight methyl cellulose as a binder; and
combinations thereof.

3. A bioresorbable vascular closure device according to claim 1, wherein the sleeve, the catch arm, and the locking sleeve each have a resorption time of 4-8 weeks in vivo.

4. A bioresorbable vascular closure device according to claim 1, further comprising:
a hemostatic plug formed of a bioresorbable material.

5. A bioresorbable vascular closure device according to claim 4, wherein the hemostatic plug bioresorbable material comprises compressed starch with 20% by weight methyl cellulose as a binder.

6. A bioresorbable vascular closure device according to claim 1, wherein the sleeve comprises an upper cylindrical portion and a lower saddle portion, the saddle portion extending radially outward from the cylindrical portion and including two diametrically opposed longitudinal slots.

7. A bioresorbable vascular closure device according to claim 6, wherein the sleeve further comprises a two diametrically opposed curved cutouts positioned circumferentially between said slots.

8. A bioresorbable vascular closure device according to claim 1, wherein the catch arm comprises an upper cylindrical portion and two diametrically opposed, longitudinally extending arms.

9. A bioresorbable vascular closure device according to claim 8, wherein each of the arms includes a lower end including a radially inwardly oriented hook.

10. A bioresorbable vascular closure device according to claim 1, wherein the locking sleeve comprises a hollow cylindrical body and two diametrically opposed, longitudinally extending ribs on an exterior surface of said body.

11. A bioresorbable vascular closure device according to claim 10, wherein the locking sleeve further comprises a two diametrically opposed curved cutouts positioned circumferentially between said ribs.

12. A method of maintaining access to the interior of a blood vessel of a patient, the method comprising:
positioning a hollow sleeve over an opening formed in the blood vessel;
positioning a pair of arms around the sleeve and around the blood vessel, each of the arms including a hook on a lower end thereof; and
positioning a locking sleeve over the hollow sleeve and the pair of arms, the locking sleeve forcing the pair of arms radially inwardly and the hooks to ensnare the blood vessel.

13. A method according to claim 12, further comprising:
adhering at least the hollow sleeve to the blood vessel.

14. A method according to claim 12, wherein the hollow sleeve, the pair of arms, and the locking sleeve are formed of at least one bioresorbable material, and further comprising:
leaving at least one subcomponent selected from the group consisting of the hollow sleeve, the pair of arms, and the locking sleeve in vivo for a time sufficient for said at least one subcomponent to be resorbed by the patient.
